(19) United States
(12) Patent Application Publication
(10) Pub. No.: US 2019/0189250 A1
(43) Pub. Date: Jun. 20, 2019

(54) Systems and Methods for Predicting and Interpreting Comprehensive Molecular Isotopic Structures and Uses Thereof

(71) Applicant: California Institute of Technology, Pasadena, CA (US)

(72) Inventor: John M. Eiler, Pasadena, CA (US)

(73) Assignee: California Institute of Technology, Pasadena, CA (US)

(21) Appl. No.: 16/221,142

(22) Filed: Dec. 14, 2018

Related U.S. Application Data
(60) Provisional application No. 62/598,721, filed on Dec. 14, 2017.

(51) Int. Cl. G16C 20/30; G16C 20/40; G16C 20/90; G16C 20/80; G01N 33/24

(52) U.S. Cl. CPC G16C 20/30; G16C 20/40; G01N 30/72; G16C 20/80; G01N 33/241; G16C 20/90

(57) ABSTRACT

Systems and methods for generating testable and quantifiable mass spectra predictions are disclosed. Generally, chemical compounds possess minute amounts of isotopes at locations within the molecule. These isotopes can affect chemical reaction kinetics and can be used to identify sources and/or information about the formation of a particular compound. Systems and methods herein obtain a chemical reaction network and chemical species and imposes constraints on the network based on chemical and reaction constants. A mass spectra is then calculated based on the reaction network, chemical species and chemical and reaction constants. A visualized mass spectra is then produced.

### CROSS REFERENCE TO RELATED APPLICATIONS

This application claims priority to U.S. Provisional Patent Application Ser. No. 62/598,721 entitled “Hypothesis Driven Predictor of Molecular Isotopic Structure and Mass Spectra,” filed Dec. 14, 2017; the disclosure of which is incorporated by reference herein in its entirety.

### FIELD OF THE INVENTION

The present invention relates to methods and systems to produce testable and quantifiable mass spectra predictions.

### BACKGROUND

Fractionations of stable isotopes by natural processes are the basis of geochemical tools used to study climate, biogeochemical cycles, and hydrology; the origin and evolution of igneous, metamorphic, and sedimentary rocks; the sources of meteorites and other extraterrestrial materials; as well as many other subjects. (See, e.g., Zachos et al. 2001 Science 292:686-93; Hedges 1991 Mar. Chem. 39:67-93; Dansgaard 1964 Tellus 16:436-68; Eiler 2001 Rev. Mineral. Geochem. 43:319-64; Clayton 2007 Annu. Rev. Earth Planet Sci. 35:1-19; the disclosures of which are incorporated herein by reference in their entirety.) The precise and accurate methods developed by earth scientists to study subtle natural isotopic variations have led to advances in the use of isotopes in forensics, biomedical science, chemistry, and other disciplines beyond the earth sciences. (See McKinney et al. 1950 Rev. Sci. Instrum. 21:724-30; see, e.g., Ehleringer et al. 2008 Proc. Natl. Acad. Sci. USA 105:2788-93; the disclosures of which are incorporated herein by reference in their entirety.) Nevertheless, most of stable isotope geochemistry is based on relatively simple measurements of bulk isotopic composition—an inventory of the proportions of isotopes in a sample, irrespective of their positions within molecular structures or the spatial relationships of rare isotopes with respect to each other.

Measurements of the distributions of isotopes in natural materials can provide a diverse, complex, and specific record of their origins, sources, and histories. A chemical compound can have an isotope substituted at various positions in its structure, which are symmetrically nonequivalent. Each symmetrically nonequivalent isotopic variant of a molecular structure is unique with respect to its chemical and physical properties (e.g., mass, intramolecular vibration frequencies, moment of inertia, and polarizability). Additionally, some compounds or species can be multiply substituted (e.g., doubly substituted or triply substituted), which increases the amount of possible isotopic versions (“isotopologues”) that exist for a particular compound. Multiply substituted species are also considered “clumped” species. Many of the possible isotopologues for a given compound exist in parts per million scales and are within the reach of modern methods of stable isotopic analysis. (See Eiler & Schauble 2004 Geochim. Cosmochim. Acta 68:4767-77, the disclosure of which is incorporated herein by reference in its entirety.) Therefore, all such species generally exhibit variations in relative concentration due to physical, chemical, and biochemical fractionations. Thus, patterns of isotopic substitution—the mix of singly and multiply substituted isotopologues that make up a sample's comprehensive molecular isotopic composition (e.g., the sample's isotopic “anatomy”)—can provide a distinctive forensic fingerprint, constraints on the sources of substrates from which the molecule was synthesized, information regarding the reaction pathways of synthesis, the temperature of formation, the geographic location of synthesis, and perhaps other information. (See Benson et al. 2006 Anal. Chem. 78:8406-11; Hattori et al. 2011 J. Agric. Food Chem. 59:9049-53. See, e.g., Monson & Hayes 1982 Geochim. Cosmochim. Acta 46:139-49; Wang et al. 2004 Geochim. Cosmochim. Acta 68:4779-97; Ehleringer et al. 2008 Proc. Natl. Acad. Sci. USA 105:2788-93; the disclosures of which are incorporated herein by reference in their entirety.) Virtually none of the isotopic diversity theorized to exist in natural molecular structures has ever been observed through conventional measurements of bulk isotope abundance ratios.

As an example of the chemical processes discussed above, FIG. 1A reveals a familiar organic compound: table sugar (sucrose; C₁₂H₂₂O₁₁). The rare-for-common substitution of isotopes (e.g., ¹³C for ¹²C) can take a variety of forms: Approximately 10% of natural sucrose contains a single ¹³C in one of its 12 carbon positions, and because all the C sites are symmetrically nonequivalent, each of the 12 possible singly ¹³C-substituted species is unique. Approximately 0.3% of natural sucrose contains at least one deuterium and ˜2% contains at least one ¹⁸O. Roughly 0.03% contains both a ¹³C and a D, and there are 264 geometrically different ways in which this double substitution can be accomplished. When adding up all possible combinations and spatial configurations of stable isotopes in sucrose, there are ˜3×10¹⁵ isotopologues of a sucrose molecule. Even though many of these combinations are exceedingly rare (some may not even exist in nature), a very large number of singly, doubly, and triply substituted versions have measurable concentrations in the range of parts per million or more—within the reach of modern methods of stable isotopic analysis. (See, e.g., Neubauer, et al., 2018 Inter. J. Mass Spec 434:276-86; Eiler, et al., 2017 Inter. J. Mass Spec 422:126-42; Eiler, et al., 2017 Geol. Soc. London 468:53-81; Eiler & Schauble 2004 Geochim. Cosmochim. Acta 68:4767-77, the disclosure of which is incorporated herein by reference in its entirety.) A full accounting of the isotopic composition of a sample of table sugar, with consideration of only those one-to-severally substituted species that seem potentially analyzable, could involve the most complex isotopic measurement ever attempted.

Many compounds undergo a series of reaction for formation, with each reaction, the products can have numerous singly or doubly substituted chemical species, where an atom is substituted for an isotopic version of that atom. For each reaction, each isotopologue must be considered to determine the effect on the reaction chain. As an example, the tricarboxylic acid cycle (TCA cycle or citric acid cycle), produces numerous compounds through its process. Each compound in the TCA cycle can have approximately 100 singly or double substituted species, which amounts to approximately 1,000 isotopic species in total for the TCA cycle.

FIG. 1B illustrates a challenge for understanding the chemical-physics behind the vibrational energies for a range of elements, molecules, sites, and states of matter. Specifically, where the vibrational energy increases with the isotopic variation. Additional examples, discussion, and disclosure of the factors that affect isotopologue formation and fractionation can be found in Eiler 2013 Annu. Rev. Earth Planet. Sci. 41:411-41, the disclosure of which is incorporated herein by reference in its entirety. Currently, no systems exist to identify actionable information arising from molecular isotopic structures.

### SUMMARY OF THE INVENTION

Systems and methods for predicting and interpreting comprehensive molecular isotopic structures in accordance with embodiments of the invention are disclosed.

In one embodiment, a system to generate isotopic structure and mass spectra predictions includes a processor, and a memory, where the memory contains instructions that when executed by the processor direct the processor to obtain a reaction network and a plurality of chemical species, where the reaction network includes at least one chemical reaction, and each chemical specie in the plurality of chemical species is a chemical compound, impose constraints on the plurality of chemical species and the reaction network, where the constraints are obtained by querying a database of reaction constants and chemical constants, calculate a mass spectra prediction based on the reaction network, chemical species, and constraints, and produce an isotopic structure prediction and a visualized mass spectrum prediction based on the calculated mass spectra prediction.

In a further embodiment, the chemical constants include constants for a plurality of chemical species, where the constants for a plurality of chemical species include at least one of the group consisting of number of atoms in the plurality of chemical species, type of atoms in the plurality of chemical species, 13 factors for the plurality of chemical species, number of bonds in the plurality of chemical species, type of bonds in the plurality of chemical species, and kinetic isotope effect for the plurality of chemical species.

In another embodiment, the reaction constants include constants for plurality of chemical reactions, where the constants include at least one of the group consisting of K_{eq}, type of reaction, rate law constants.

In a still further embodiment, the reaction network contains a domain, where the domain represents a physical space having at least one physical property, where the at least one physical property is selected from the group consisting of specified volume, surface area, temperature, pressure, pH, Eh, and oxygen fugacity.

In still another embodiment, the instructions also direct the processor to impose initial constraints on the reaction network based on activity of the plurality of chemical species.

In a yet further embodiment, the instructions also direct the processor to impose initial constraints on the reaction network based on initial abundance of the plurality of chemical species.

In yet another embodiment, the instructions also direct the processor to query a user whether to run a time-varying solution or steady state solution, and impose a further constraint to change the abundance of the plurality of chemical species over time.

In a further embodiment again, the instructions also direct the processor to query a database of equilibrium partition functions to identify specific equilibrium partition functions for the plurality of chemical species, wherein the partition functions define equilibrium proportions of singly and doubly substituted isotopologues of various chemical species.

In another embodiment again, the instructions also direct the processor to calculate an equilibrium partition function for at least one of the plurality of chemical species.

In a further additional embodiment, the instructions also direct the processor to obtain initial isotopic contents for the plurality of chemical species, combine the initial isotopic contents with the abundance of the plurality of chemical species, calculate isotope exchange equilibria for the plurality of chemical species based on the specific partition functions and the combined initial isotopic contents and the abundance of the plurality of chemical species, determine kinetic isotope effects of the plurality of chemical species, and determine proportions of isotopologues of the plurality of chemical species.

In another additional embodiment, the instructions also direct the processor to query a database of mass spectra to identify a mass spectra for each of the plurality of chemical species, calculate a mass spectra prediction based on the identified mass spectra and the proportions of isotopologues, and generate the visualized mass spectra prediction based on the calculated mass spectra prediction.

In a still yet further embodiment, the instructions also direct the processor to define a mass resolution of the mass spectra prediction, and recalculate the mass spectra prediction based on the defined mass resolution.

In still yet another embodiment, the instructions also direct the processor to query a database of reference standards to identify a reference standard compatible with the identified mass spectra, and produce the compatible reference standard.

In a still further embodiment again, the visualized mass spectra prediction is generated by ratioing the calculated mass spectra prediction to a mass spectrum of the compatible reference standard.

In still another embodiment again, the system also includes a graphical user interface for accepting input from a user and producing the visualized mass spectra prediction.

In a still further additional embodiment, the system also includes four graphical user interfaces, where a first graphical user interface is used to input the reaction network, a second graphical user interface is used to input the plurality of chemical species, a third graphical user interface is used to output the isotopic structure prediction, and a fourth graphical user interface is used to output the visualized mass spectrum prediction.

In still another additional embodiment, the obtained reaction network and the obtained plurality of chemical species are received over a network from a user device.

In a yet further embodiment again, the isotopic structure prediction and the visualized mass spectrum prediction are provided to a user device over a network.

In yet another embodiment again, a method of extracting oil includes obtaining a mass spectrum from a sample obtained from a geologic source to identify at least one compound present in the sample, defining a reaction network to synthesize the at least one compound present in the sample and a desired compound, where the reaction network contains a domain possessing a physical property which has multiple settings, generating a plurality of visualized mass spectra predictions based on the reaction network, where the plurality of visualized mass spectra predictions represent the reaction network at each of the multiple settings of the physical property, identifying a first setting and a second setting from the reaction network, where the first setting identifies a physical property condition that led to synthesis of the desired compound and the second setting identifies a physical property condition that led to synthesis of the at least one compound present in the sample, quantifying a difference in the at least one physical property that led to synthesis of the desired compound over the at least one compound present in the sample, and extracting the desired compound based on the quantified difference.

In a yet further additional embodiment, the extracting step is accomplished by one of the group consisting of mining, drilling, and fracking.

### BRIEF DESCRIPTION OF THE DRAWINGS

These and other features and advantages of the present invention will be better understood by reference to the following detailed description when considered in conjunction with the accompanying drawings where:

FIG. 1A illustrates a sucrose molecule demonstrating various isotope substitutions in accordance with various embodiments.

FIG. 1B illustrates the effect of isotopes on different molecular scenarios, in accordance with various embodiments.

FIG. 2 illustrates a network diagram of systems in accordance with various embodiments.

FIG. 3 illustrates a system configuration in accordance with various embodiments.

FIG. 4 illustrates a method to produce mass spectra predictions in accordance with various embodiments.

FIG. 5 illustrates a method to identify equilibrium partition functions in accordance with various embodiments.

FIG. 6 illustrates a method to determine isotopologue proportions in accordance with various embodiments.

FIG. 7 illustrates a method to generate visualized mass spectra predictions in accordance with various embodiments.

FIGS. 8A and 8B illustrate a method to test a hypothesis in accordance with various embodiments.

FIG. 9 illustrates a method to identify a source of a compound in accordance with various embodiments.

FIG. 10 illustrates a method to extract a natural resource in accordance with various embodiments.

FIG. 11 illustrates user interface including input of a network reaction in accordance with various embodiments.

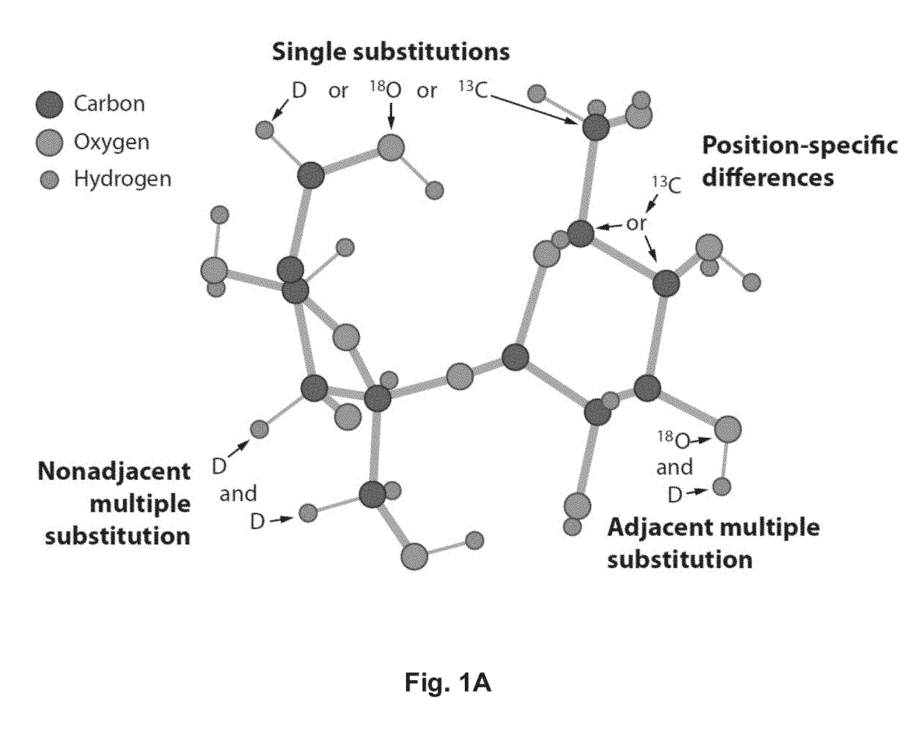

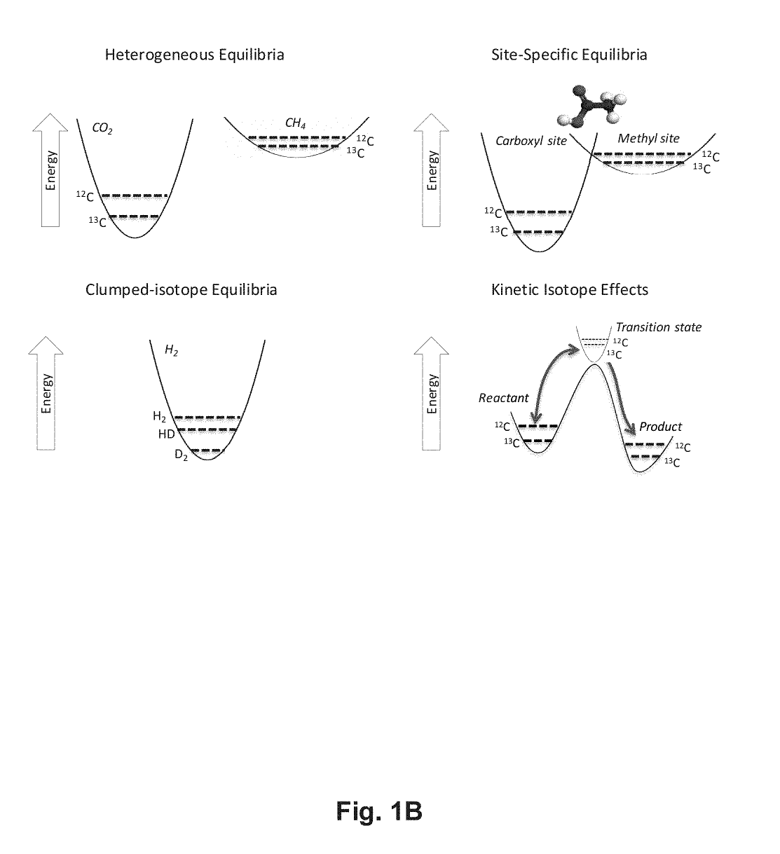

### DETAILED DESCRIPTION

Turning now to the drawings, systems and methods for predicting and interpreting comprehensive molecular isotopic structures and uses thereof in accordance with many embodiments of the invention are illustrated.

The effect of isotopic structure on kinetics follows specific, physical rules according to the specific reaction conditions in which the reaction takes place. By following these rules, resultant compounds contain molecular isotopes unique to the specific reaction, which reveal information about the reaction kinetics, much like a fingerprint. This fingerprint can be utilized for many purposes, such as in natural resource extraction, forensics, and archaeology.

In numerous embodiments, users define hypothesized reaction networks (e.g., a group of one or more reactions among co-existing chemical species) translate those reaction networks into predictions of the proportions of all singly and doubly substituted isotopologues of all chemical species in the model, as a function of time in cases where the system is not in steady state. Finally, certain embodiments present the user with predicted mass spectra of each chemical species and identifies which features of the mass spectrum contrast most strongly with reference materials and/or change the most over the course of the reaction network's evolution. Thus, many embodiments produce a hypothesized chemical process which can be translated into explicit, quantitative, and testable predictions regarding the evolution of measurable features of the mass spectrum of any reactant or product species.

The explicit, quantitative, and testable predictions of chemical structure produced by various embodiments, improve such fields as natural resource extraction, forensics, and archaeology by identifying sources and origins of chemical compounds, environmental characteristics present during the formation of the chemical compounds, including during different geologic eras or historic time periods, as well as identifying indicia of better sources for a desired compound.

In many embodiments, reaction networks are defined and chemical species that participate as reactants or products in the network are selected. In such embodiments, reactions networks include model domains, chemical species, and reactions to define their hypothesized reaction networks. In these embodiments, a domain represents a physical space. As a representative of a physical space, a domain in various embodiments comprises a specified volume and surface area and containing one or more chemical species. All potentials, such as temperature, pressure, and activities of chemical species, are assumed to be uniform across a given domain at a given time. Examples of a domain including a cell, a volume of water, such as a deciliter, or the lower boundary layer of the earth's atmosphere. In some embodiments, domains have physical properties, including specified temperature, pressure, and/or chemical potentials, such as pH, Eh, and/or oxygen fugacity.

Additionally, a chemical species is any atomic or molecular compound, either neutral or charged, either in a ground or excited electronic state that is present in one or more domains and participates in one or more reactions. Examples of chemical species, include helium atoms, water molecules, acetate molecules, oxygen radicals, or hydrogen ions.

Further, reactions are relations between two or more chemical species that can be expressed as equations that follow principles of mass balance. In various embodiments, reactions are defined as reversible equilibrium or irreversible reactions. A reversible equilibrium is a reaction with no net change over time in the proportions of reactants and products. Reversible equilibria typically have rates of forward and back reactions that are much faster than the rates of rate limiting reactions in a reaction network. An example of reversible equilibrium includes the isotope exchange of ¹⁸O for ¹⁶O between a carbonate ion and a bicarbonate ion, in a system that is simultaneously undergoing a slower, irreversible reaction such as dehydroxylation of the bicarbonate ion. Reversible equilibria can apply to all molecular sites and isotopologues of a chemical species, or reversible equilibria can be specified to apply only to isotopologues of one site or group of atomic sites in a molecule. For example, various embodiments can specify that carboxyl sites (CO₂H groups) of alanine are in an oxygen and hydrogen isotope exchange equilibrium with respect to H₂O in aqueous solution, but carbon in those carboxyl groups do not participate in carbon isotope exchange with any co-existing species. Information about reversible equilibria can be included in a database of chemical and reaction constants. Further, irreversible reactions include reactions that irreversibly transform reactant to product. Irreversible reactions are defined to be of a specific type, choosing form a list of reaction types. Examples of irreversible reactions include hemolytic cleavage, beta scission, hydration, dehydration, hydroxylation, dehydroxylation, hydrogenation, dehydrogenation, carboxylation, decarboxylation, amination, deamination, oxidation, reduction, and proton transfer. Irreversible reactions occur at rates of moles reactant consumed per unit time (a molar flux), which can be specified or calculated using a database of chemical and reaction constants or may vary over time as part of the reaction network solution.

Many embodiments will include one or more databases of chemical and reaction constants. Chemical species and reactions are associated with a set of material constants that are relevant to the model of a reaction network. All such constants that relate to isotopic constants, distributions, and fractionations are described by one or more databases. One type of database is a database of chemical species constants, which can include any information describing a chemical species, including molecular structures, atomic and molecular weights, densities, activity/composition relations, and equations of state. Another database is a reaction constant database, which can include information describing chemical reactions, including equilibrium constants, rate constants, solubilities, phase transition temperatures, vapor pressure curves, and diffusion coefficients.

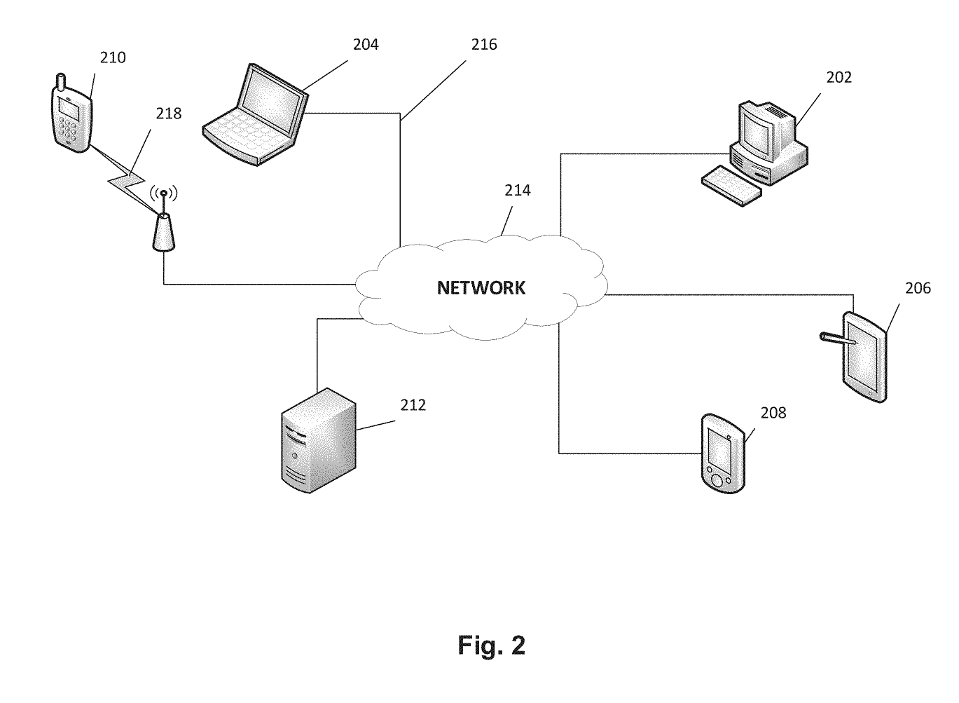

Many embodiments for predicting and interpreting molecular isotopic structures in accordance with many embodiments include computing devices configured to compute predicted structures and mass spectra generated by isotopologues generated by chemical reactions under various reaction conditions. FIG. 2 illustrates possible configurations of computing devices used in various embodiments, such that user computing devices, including a desktop computer 202, laptop computer 204, tablet 206, personal digital assistant 208, and cell phone 210 are configured to operate locally, such that data processing occurs on the same computer that inputs are entered and/or manipulated. FIG. 2 also illustrates a networked configuration, where the various user computing devices (202, 204, 206, 208, and/or 210) are connected to a processing server 212 through a network 214 through wired 216 or wireless 218 means. In a networked configuration, user computing devices of some embodiments are used to input data, which is transmitted to a processing server 212, which performs data processing and returns the results to the user computing device(s) where inputs were entered. Embodiments in a networked configuration allows for updates (such as processing improvements, database improvements, and/or database updates) to occur in a central location, rather than requiring updates to be performed on numerous individual user computing devices. Further embodiments are configured to operate in both local and networked configurations, such that processing intensive operations are performed at a processing server (e.g., 212), while less processing intensive operations occur locally on a user computing device (e.g., 202, 204, etc.).

### Systems of Operation

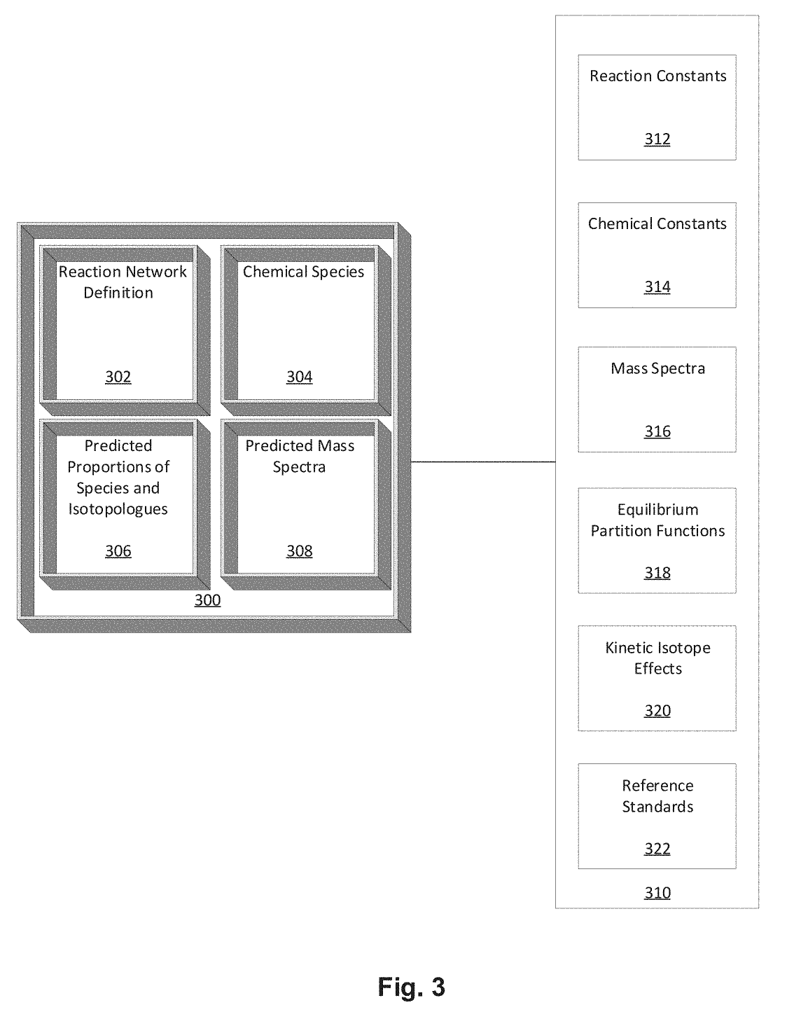

FIG. 3 illustrates computing systems in accordance with various embodiments. Computing systems of some embodiments include one or more user interfaces 300 configured to input parameters and/or receive output and one or more databases 310. In some embodiments, the database 310 will comprise a single database of reaction constants, chemical constants, mass spectra, equilibrium partition functions, kinetic isotope effects, and reference standards. Additional embodiments will possess individual databases, such that these embodiments will possess a database of reaction constants 312, a database of chemical constants 314, a database of mass spectra 316, a database of equilibrium partition functions 318, a database of kinetic isotope effects 320, and/or a database of reference standards 322. In some embodiments, the user interface or interfaces will be a graphical user interface or interfaces. In various embodiments, such as illustrated in FIG. 3, a first user interface 302 is used to define a reaction network. In this reaction network definition interface 302, a user selects and manipulates graphical symbols for model domains, chemical species, and reactions to define a hypothesized reaction network. In various embodiments, a user will define reactions using a tool set that represents reversible equilibria and each type of irreversible reaction using unique symbols, which are linked to the relevant reactant and product chemical species. In various embodiments, a user is allowed to define and name a new reaction type by specifying the chemical species that participate as reactants and products and the bonds that are broken and/or formed when a reactant is transformed into a product. In some embodiments, unique symbols are linked to specific sites and bonds within each reactant and product chemical species. In various embodiments, a user can define a single domain in which all reactions takes place, while some embodiments allow a user to define reactants interacting with products in at least one other domain.

Various embodiments include a second user interface 304, which allows a user to manipulate symbols for atoms and chemical bonds to construct new chemical species that are not previously part of the one or more databases 310. In various embodiments, a user will select chemical species that participate as reactants or products in the reaction network by either selecting the chemical species form a searchable menu or defining them using a graphical tool that permits the user to draw chemical compounds using a tool set of atom and bond types. In various embodiments, new chemical species defined by a user will be added to the one or more databases 310. In various embodiments, chemical species are defined to have specified molar amounts in each domain, which can be interconverted with concentrations, molarities, and chemical activities using data in the database of chemical and reaction constants 310 or in individual databases such as chemical and reaction constants 310. In some embodiments where a chemical species is present in more than one domain, the chemical species is entered multiple times. In some of the embodiments where a chemical species is in more than one domain, each entry will have the same initial activity, while in other embodiments, each entry will have different initial activities.

Certain embodiments further include one or more output interfaces (e.g., 306 and 308). In various embodiments, a third interface 306 to output predicted proportions of chemical species and their isotopologues as tables, figures, and animations, and a fourth interface 308 outputs predicted mass spectra of chemical species as tables and figures. In various embodiments, the fourth interface annotates the output with automated recommendations regarding the best targets for mass spectrometric measurements that will test the validity of the user-defined reaction network, such as the reaction network defined in the reaction network interface 302.

While the reaction network definition interface 302, the chemical species interface 304, predicted proportions interface 306, and predicted mass spectra interface 308 are described separately, some embodiments will allow a user to input reaction networks and chemical species and provide output in the form of predicted proportions and mass spectra into a single interface. Additionally, some embodiments will provide two interfaces, where one interface allows a user to input reaction networks and chemical species, while a second interface provides output in the form of predicted proportions and mass spectra. Further, some embodiments will utilize a single interface input reaction networks and chemical species and a second interface to output predicted proportions, and a third interface to output mass spectra. Similarly, some embodiments will utilize one interface in input reaction networks, a second interface to input chemical species, and a third interface to output predicted proportions and mass spectra.

### Computation of Reaction Network Output

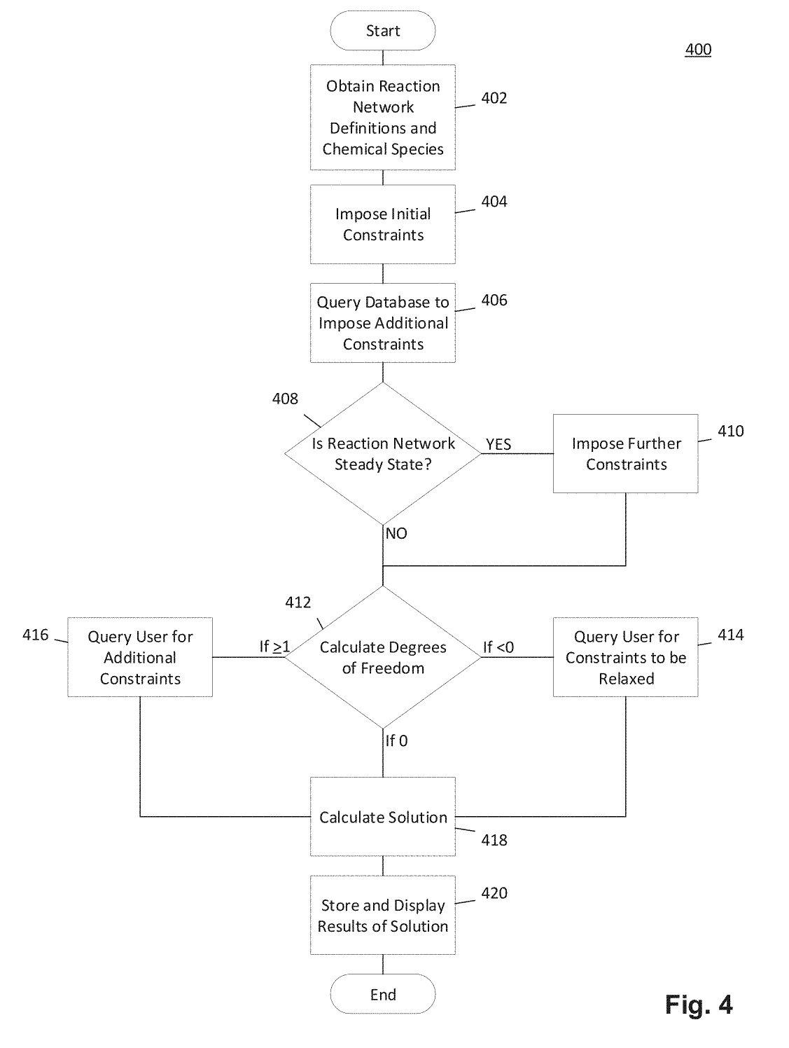

FIG. 4 illustrates a process 400 to produce the predicted proportions and mass spectra from reaction networks and chemical species, which includes a number of steps utilized in various embodiments. Certain embodiments are directed to systems configured to execute the process 400. In various embodiments, Step 402 involves obtaining reaction network definitions and chemical species. In certain embodiments, the reaction network definitions and chemical species are received from a user, such that one or more users inputs chemical reactions into a system. In some embodiments, these inputs will use interfaces, such as those described above in relation to FIG. 3 (e.g., 300, 302, and 304). Each species in the reaction network possesses a number of variables, including the total inventory of each isotope tracked, including all site-specific single substitutions, double substitution types, and triple substitution types. In certain embodiments, all other substitution types are stochastically defined. In various embodiments, end-member reactants are required, and in some embodiments, other states are permitted to be set. Further, variables per reaction are also set in some embodiments, including the type of reaction (e.g., equilibrium or irreversible). In an equilibrium reaction, the Ke_{q} of molecular and side-by-side variables are input. For irreversible reactions, the type of bond breaking (e.g., hemolytic cleavage and β-scission) are input along with Rax values and kinetic isotope effect (KIE) values.

At Step 404 of certain embodiments, various embodiments of this process will impose initial constraints on the quantities of each chemical species present in each domain. In some embodiments, the initial constraints will be specified as zero (initially absent), semi-infinite (present at a stipulated concentration or activity, which remains constant over time in the reaction network model), or some specified initial concentration or activity, which is allowed to vary over time. In certain embodiments, closure is applied as a constraint, such as when the sum of concentrations of all specified chemical species equals some value. In various embodiments this value is set to 1.

At Step 406 of some embodiments, the process 400 queries a chemical and reaction database (e.g., FIG. 3, 310) to impose additional constraints that are relevant to mass balance at each reaction in the defined reaction network. Examples of these constraints include equilibrium constants and rate constants of reactions. In additional embodiments, a user can override the constraints imposed by the database with custom constraint values. Chemical constant databases of certain embodiments contain one or more of the following information: number and type of atoms, 13 factors, bonds (including clumping K_{eq} and kinetic isotope effect (KIE) for each reaction type). Reaction constant databases include K_{eq} for equilibrium equations and type and rate law for irreversible reactions, in some embodiments.

At Step 408 of various embodiments, the process 400 queries the user regarding whether the user desires a time varying or steady state solution. If the user seeks a steady state solution, the process 400 proceeds to Step 410, while a time varying solution will proceed to Step 412. It should be noted that certain embodiments will perform Step 408 at a different time, such that a user is queried simultaneously with or immediately after defining a reaction network and chemical species of Step 402.

If a user desires a steady state solution, some embodiments will impose a further constraint that the change over time of each chemical species abundance and reaction rate will be zero at Step 410. Whether a user desires a steady state or time varying solution, various embodiments of the process 400 will adjust the degrees of freedom to zero at steps 412, 414, and 416. Specifically, various embodiments of this process calculate degrees of freedom for each reaction at Step 412. In certain embodiments, the degrees of freedom is equal to the number of independent chemical species minus the constraints.

At Step 412, if the degrees of freedom are less than zero, then various embodiments will query the user to relax one or more constraints at Step 414, such that the degrees of freedom will increase. In additional embodiments, if there are more than one degree of freedom, the user is queried for additional constraints at Step 416, such as reaction rates, branching ratios, equilibrium constants, or amounts of chemical species, such that the degrees of freedom will decrease.

When the degrees of freedom reach zero, or if the degrees of freedom are zero at Step 412, the process 400 of certain embodiments will calculate the time varying or steady state solution via the family of constraining equations for each reaction—for example, the rates of production equal the rates of consumption for each species. If the user desired a time varying solution, such as at Step 408, Step 418 will calculate the solution with a specified time step and total model duration in various embodiments.

At Step 420, the process 400 of various embodiments will store and/or display the results of the solution. This step 420 in certain embodiments will be performed using a user interface, such as illustrated in FIG. 3 (e.g., 300, 306, and 308). In certain embodiments, these results represent the quantitative realization of the user's hypothesized reaction network. At this point, the output of various embodiments represents a fully defined reaction network hypothesis, and additional embodiments are ready to examine implications of the hypothesis for isotopic contents and structures of reactants and products, either at steady state or each time step of the model.

### Equilibrium Proportions of Molecular Isotopologues

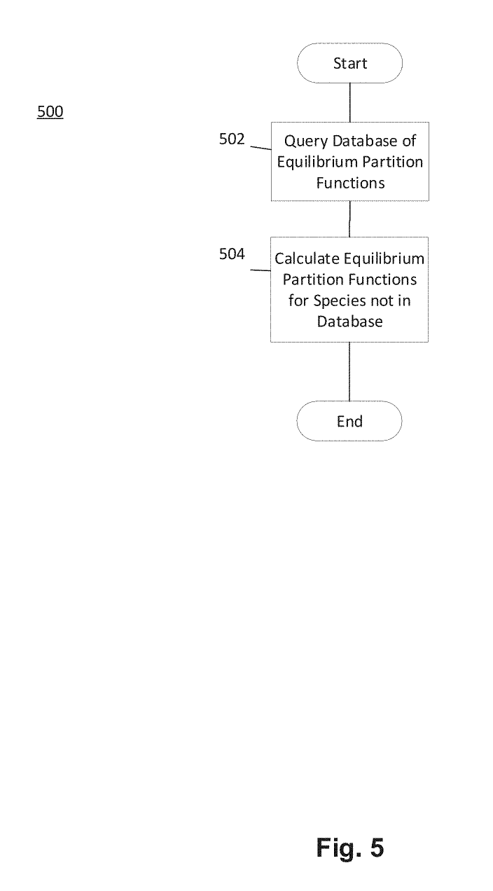

Upon completion of a hypothesized reaction network, such as generated in process 400, certain embodiments generate equilibrium partition functions for isotopologues of the various chemical species in the model. FIG. 5 illustrates a process 500 by which equilibrium proportions are generated for chemical species in the model. In various embodiments, the process 500 is accomplished by a computing system configured to execute the process.

The propensity of atomic sites to concentrate heavy isotopes at thermodynamic equilibrium is described by the partition function ratio of an isotopologue containing a rare isotope at that site, or for clumped isotopologues, at sets of sites. Once a hypothesized reaction network is fully defined, process 500 of various embodiments will query a database of such equilibrium partition functions at Step 502. The partition functions in the partition function database define the equilibrium proportions of all singly and doubly substituted isotopologues of each chemical species in the model. If some chemical species are not present in the database, additional embodiments will calculate the equilibrium partition functions based on molecular structure at Step 504. In certain embodiments, these equilibrium partition functions are calculated using a structural activity relationship type model in which the partition function is parameterized as a function of structural characteristics of the molecule.

### Defining Initial Constraints on Isotopic Contents

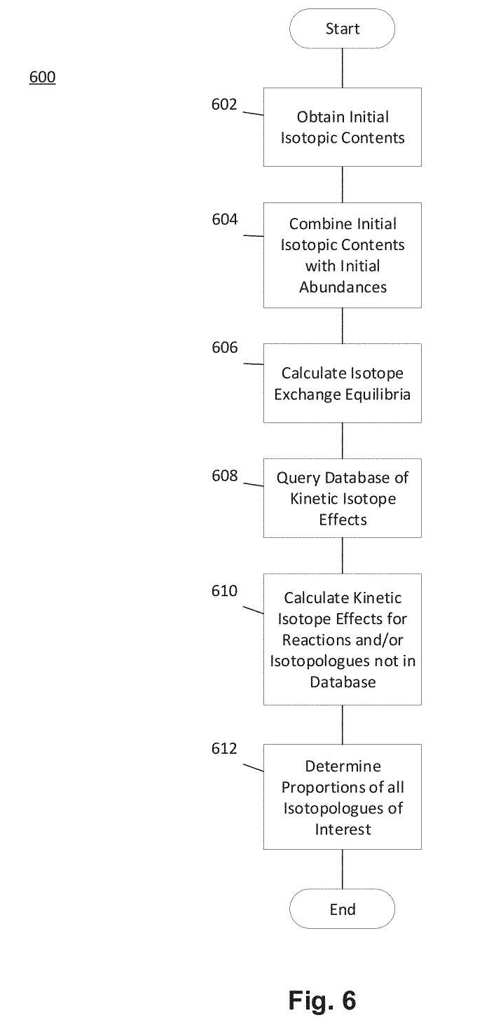

Several embodiments will utilize process 600 illustrated in FIG. 6 to calculate inventories of isotopologues that are present in the model domains at the start of the model's time frame. Certain embodiments are directed to computing systems configured to execute the process 600. In such embodiments, process 600 will obtain initial isotopic contents and structures of all chemical species that are present in the reaction network model as part of an initial condition at Step 602. At this step, each compound will be assigned a bulk (molecule average) isotopic content for each element present in that compound's chemical formula. In these embodiments, the bulk isotopic content can be measured in any of several commonly used units, such as isotope abundance ratio, difference in isotope ratio between the compound of interest and a reference standard, or molar concentration of specified isotopologues. In certain embodiments, Step 602 queries a user to choose among three options, including random, equilibrium, and user specified. The random option of some embodiments provides for a statistical distribution of all isotopes across all relevant atomic sites. The equilibrium option of additional embodiments provides for an equilibrium based on equilibrium partition functions and the initial temperature of the domain in which the chemical species is found. In further embodiments, the user specified option presents the user with a random isotopic structure for that species and freely increases or decreases proportions of isotopologues, where the bulk isotopic content is recalculated after each change. Once a user defines the isotopic contents and structures of chemical species that are initially present at the start of the model's time duration, the user input is combined with the initial abundances of chemical species in each domain at Step 604. At Step 604 of some embodiments, this combination is used to calculate the inventories of all isotopologues that are present in the model domains at the start of the model's time frame.

### Computation of Exchange Equilibria

All specified exchange equilibria have an associated isotope exchange equilibrium constant. After calculating the inventories of isotopologues present in the reaction network, various embodiments include Step 606 to calculate isotope exchange equilibrium constants for the chemical species in question using standard statistical thermodynamic theory based on the partition functions of the isotopologues of the chemical species in question.

### Determining Kinetic Isotope Effects

In additional embodiments, process 600 further includes a step 608 to query a database of kinetic isotope effects associated with all irreversible reactions and mass transport process in the reaction network model. In various embodiments, the database of kinetic isotope effects is part of an existing database, such that the database of kinetic isotope effects in included in a single database of reaction and chemical constants, a database of only chemical constants, or in a database of reaction constants. In embodiments where the defined reaction network involves reactions and/or isotopologues for which kinetic isotope effects are not known, the kinetic isotope effects are calculated based on the molecular structures of the reactants and products and user-specified identification of bonds being broken and/or formed at Step 610. At Step 610 of various embodiments, the kinetic effects are calculated using an algorithm that scales kinetic isotope effects as functions of the partition functions of the reactant and product species, which are used to generate approximations of the partition functions of the reaction transition states, such as in Step 504 of process 500, illustrated in FIG. 5.

After determining kinetic isotope effects for all reactions and isotopologues in the defined reaction network, process 600 of various embodiments proceeds to Step 612 to determine time varying and/or steady state proportions of all isotopologues of interest for all molecular species in the reaction network. In some embodiments, the reactions in the model are combined with defined isotope exchange equilibria and kinetic isotope effects to solve for proportions of isotopologues of all chemical species present at steady state or changing over time (e.g., one time step per computation). In certain embodiments, these calculations are fully defined by combination of principles of mass balance with the parameters defining the quantitative reaction network model and the specific initial inventory of isotopologues in the model.

### Generation of Predicted Mass Spectra

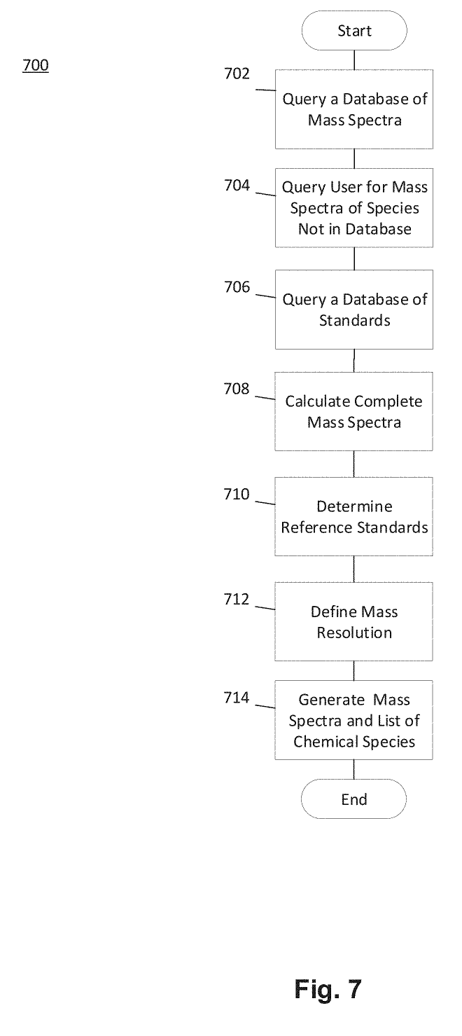

In certain embodiments, the processes described above yield a set of predicted proportions of isotopologues of all chemical species in the model, either at steady state or as time varying functions. In various embodiments, these proportions of isotopologues are translated into predicted mass spectra for each chemical species using process 700, illustrated in FIG. 7. Certain embodiments are directed to computing systems configured to execute the process 700. These predicted mass spectra of some embodiments allow the model predictions to be articulated explicitly measurable quantities. Additionally in some embodiments, these mass spectra are automatically evaluated to recommend the most useful and efficient tests of the defined reaction network.

At Step 702 of this process, various embodiments query a database to retrieve standard mass spectra for chemical species that are part of the reaction network model. The mass spectra for this database can arise from any type of mass spectroscopy available, such that certain embodiments will utilize electron impact ionization (EI), while additional embodiments will utilize electrospray ionization (ESI), and further embodiments will utilize collision cell fragmentation (MS-MS). Further embodiments will provide mass spectra for multiple types of mass spectroscopy, such that these embodiments will provide mass spectra for EI and ESI, ESI and MS-MS, EI and MS-MS, or all EI, ESI, and MS-MS.

At Step 704, some embodiments query the user to input mass spectra for chemical species not part of the database from Step 702. At this step, the user can input mass spectra as tab delimited files and/or typed input of the stoichiometries of peaks of interest. In various embodiments, these peaks are automatically converted to masses using a chemical database, such as one or more databases described above. Additionally, some embodiments will automatically convert the peaks into relative immensities. In certain embodiments, these peaks will be automatically entered into a mass spectra database, while other embodiments the peaks will undergo quality control inspection and added to a central database by an administrator, if the databases are held in a central processing server, such as described above.

At Step 706, various embodiments will query a database of standards to identify measured, estimated, or assumed isotopic contents and structures of materials that can serve as reference standards for experiments to validate or verify the defined reaction network.

At Step 708, certain embodiments will calculate complete mass spectra for all compounds of interest. This calculated mass spectra will consider all singly, double, and triply substituted isotopologues of all fragment ions in certain embodiments. In further embodiments, the calculated mass spectra are calculated by combining the proportions of isotopologues from the complete reaction network model with the mass spectrum of a compound of interest.

Once complete mass spectra are produced in Step 708, various embodiments will determine one or more reference standards to which the modeled chemical species will be compared at Step 710. In certain embodiments, Step 710 queries the user to identify the reference standard(s). In some embodiments, the reference compound is present in the database, while other embodiments will allow the user to take the initial isotopic composition of the chemical species (e.g., at time zero of the model) as a reference standard. Further embodiments allow the user to define the properties of a hypothetical standard using the hypothetical standard's bulk isotopic content and structure. Once a reference material is selected, Step 710 of various embodiments calculates a complete mass spectrum for the reference material, whether the reference compound is real or hypothesized.

At Step 712 of some embodiments, the mass resolution of the mass spectrum is defined. In various embodiments, this step is accomplished by querying the user to define the mass resolution. Once a mass resolution is defined, several embodiments will recalculate the complete mass spectra of the compound of interest and reference standard(s). In some embodiments, this recalculation will combine peaks that are unresolved at the specific resolution determined in this step.

At Step 714, various embodiments generate a visualized mass spectra and rank ordered list of greatest to small proportional contrast in ratio of chemical species. These results are accomplished by taking the ratio of the calculated chemical species mass spectra (e.g., from Step 708) to the reference mass spectrum (e.g., from Step 710) in certain embodiments. In the ordered list of several embodiments, each peak in the list is also given with its relative intensity in the modeled sample mass spectra to provide a guide to the relative difficulty of measurement.

In various embodiments, certain steps of processes 400-700, illustrated in FIGS. 4-7 respectively, may not be necessary or may be performed in a different order as described above, depending on the specific characteristics or phenomena occurring at the molecular structure. Further, these processes can be combined into a single process or kept separate to be used in a modular fashion, such that entire modules may be used in custom orders based on the need of a user.

Further, additional embodiments will include automated adjustment of the parameters defining the reaction network until the predicted reference-normalized mass spectra of one or more chemical species matches a user-input reference-normalized measurement of those mass spectra. Further additional embodiments will include automated analysis of the technical parameters required for a mass spectrometric measurement that is purpose-designed to test a hypothesized reaction network model. For example, this automated analysis can specify the ionization method, mass resolution, targeted peaks, and analytical duration required to observe the change in the mass spectrum predicted by a specific hypothesis.

### System for Hypothesis Testing

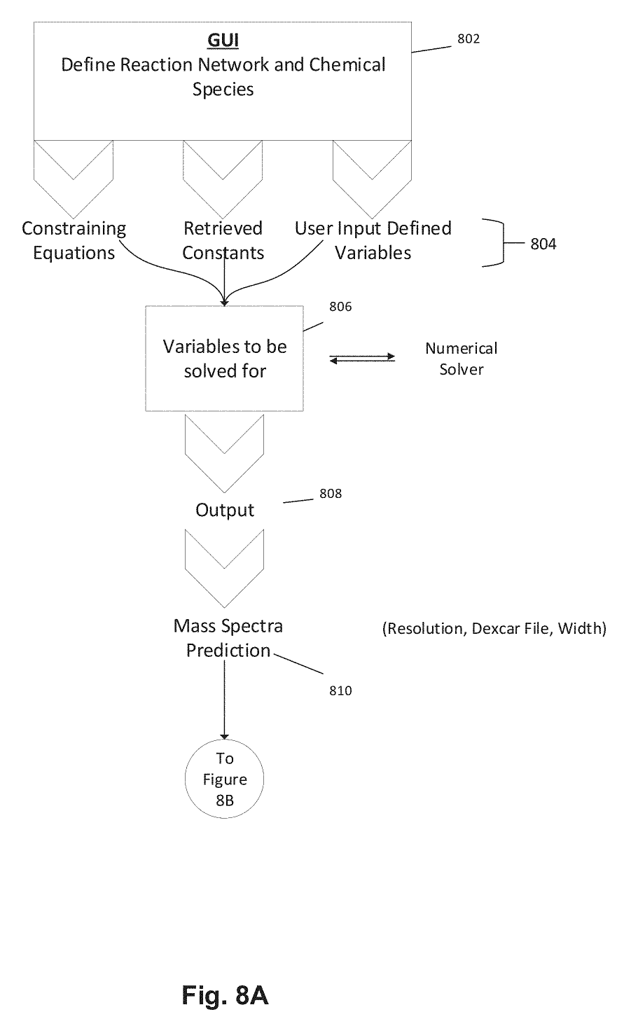

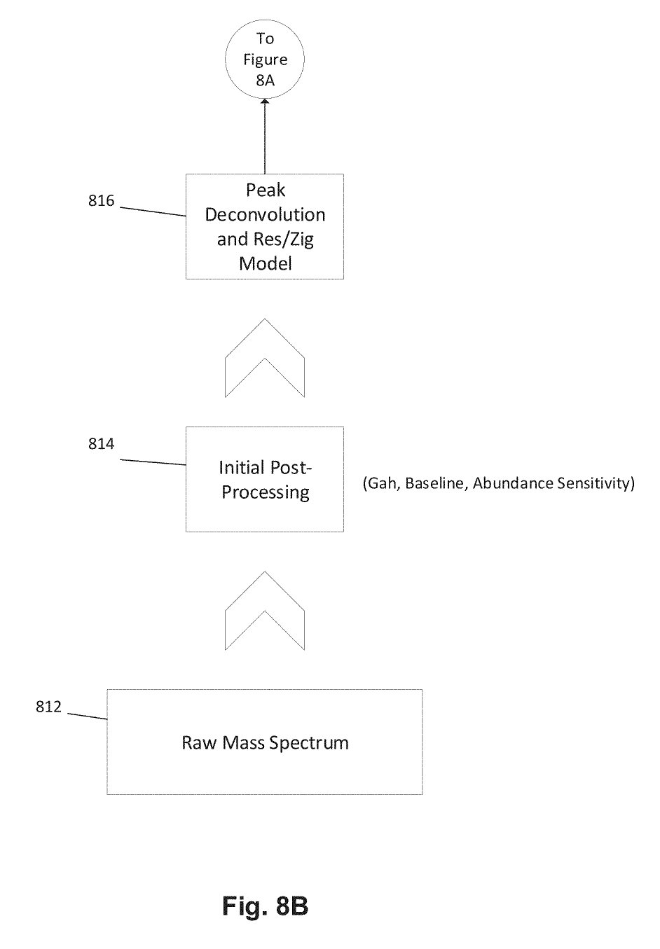

Utilizing methods described herein, certain embodiments generate testable hypothesized reaction networks, as illustrated in FIGS. 8A and 8B. Specifically, FIG. 8A illustrates a process for generating predicted mass spectra, while FIG. 8B illustrates how these results can be tested through experimental means.

In FIG. 8A, a graphical user interface (GUI) 802 for defining a hypothesized reaction in methods described herein with regard to some embodiments. Upon defining the reaction network, certain factors 804, including constraining equations, received constants from a user, and user-input defined variables are extracted to generate a set of variables 806 to be solved for using methods described herein. Once solved, these variables are output in a raw form 808, which are translated into predicted mass spectra based on the hypothesized reaction network.

Turning to FIG. 8B, certain embodiments test the hypothesized reaction network in an experimental setting. Specifically, a raw mass spectrum 812 is produced via suitable mass spectroscopy equipment. The raw mass spectrum undergoes an initial post-processing 814 through means such as Gah, Baseline, and Abundance Sensitivity to result in a finalized, peak deconvoluted model 816. This resultant mass spectrum illustrated in FIG. 8B can be compared to the predicted mass spectrum illustrated in FIG. 8A. Under this methodology, the predicted mass spectra produces an explicit, quantitative, and testable prediction regarding the evolution of measurable features of the mass spectrum of any reactant or product species.

### Application of Processes and Systems

As mentioned above, various possible applications exist for embodiments to improve such areas as forensics and natural resource extraction. Examples of how a system as described above could be used in these environments are described below:

### Forensics

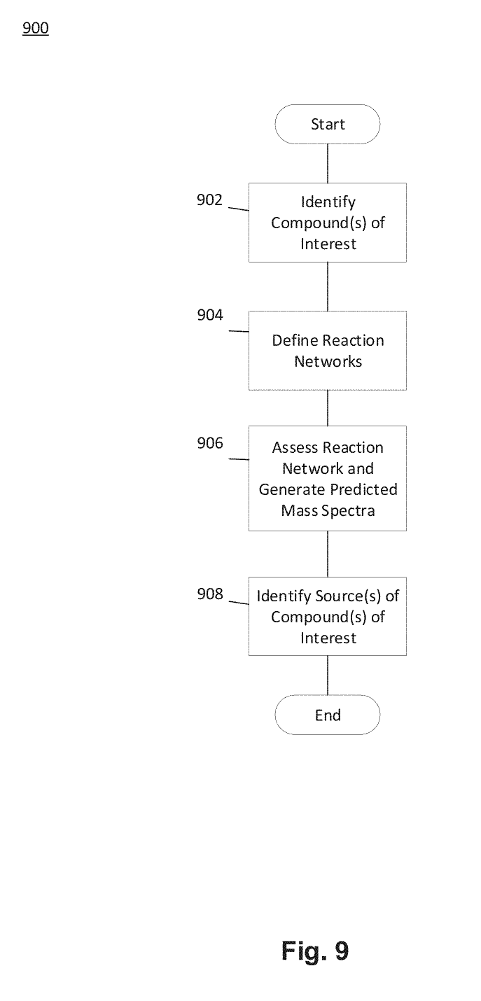

A use of systems and methods as described in this document include the use in forensic sciences. In forensics, certain users seek to identify the origin of a particular compound or compounds and/or to identify the reaction conditions that gave rise to the particular compound or compounds of interest. FIG. 9 illustrates a process 900 describing how some embodiments can be used in forensic research or analysis.

For some embodiments used in forensics, a user identifies a compound or compounds of interest at Step 902. The compounds in this step can be any compound where isotopes exist for one or more atoms, such as described within this disclosure. For example, a user could select organic molecules, such as hydrocarbons or sugars, including sucrose.

Upon identifying one or more compounds of interest, a user will define one or more reaction networks that can be utilized to synthesize the compounds of interest at Step 904 of certain embodiments. For compounds of interest that have multiple methods of synthesis, some embodiments will allow the user to enter all known or possible reaction networks, while other embodiments may only allow a single reaction network at a time for further analysis. As noted above, the reaction network includes defining domains, chemical species, and reactions utilized to synthesize the compound or compounds of interest.

At Step 906, systems and methods will assess the reaction networks by querying databases for reaction and chemical constants to generate predicted mass spectra and/or predicted proportions of chemical species and isotopologues as described herein, in various embodiments.

At Step 908, mass spectra of the compounds of interest are compared to the predicted mass spectra and/or proportions of chemical species and isotopologues, in many embodiments. In some embodiments, this step is accomplished automatically by computer systems of certain embodiments, which contain the software for generating the predicted mass spectra. This comparison can be accomplished by known means of comparing similarity and/or identity between mass spectra. Upon identifying mass spectra that show a level of similarity within confidence, the source or sources for the compound of compounds of interest will be identified by certain embodiments.

### Natural Resource Exploration

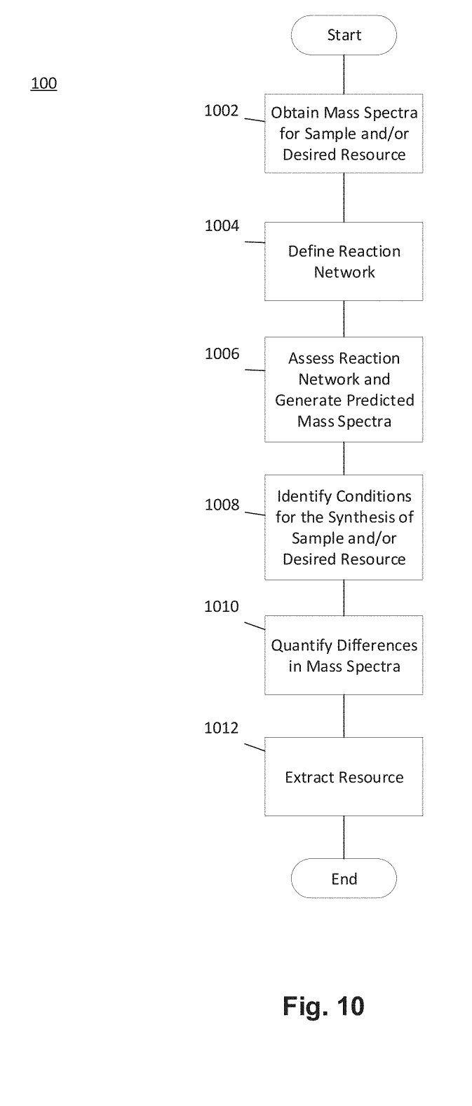

An additional use of systems and methods as described in this document include the use in natural resource extraction, an example of process 1000, which uses above methodologies in natural resource extraction is illustrated in FIG. 10. In natural resource extraction, certain users seek to better deposits of minerals and/or hydrocarbons of value. Many of the chemical reactions that result in the production of minerals (e.g., diamonds, opal, coltan, etc.) or other resources of value, such as hydrocarbons (e.g., crude oil, natural gas, etc.) require specific reaction conditions for the proper formation of these compounds. If the reaction conditions are not appropriate for the formation of these compounds, non-desired compounds form instead. Certain embodiments utilize systems and methods described herein to utilize the non-desired compounds to identify sources or locations for deposits of minerals or other resources of value.

At Step 1002 of various embodiments, a user obtains mass spectra for a desired resource. In additional embodiments, a user will also obtain mass spectra for a sample. The sample is obtained from a geographic or geologic source, where the natural resource may exist. In certain embodiments, this sample is excavated, located, or mined directly by the user or may be sent to a user from another person. In some embodiments, a user generates a mass spectra of this sample through known methods of generating a mass spectra, including EI, ESI, MS-MS, or any other known or appropriate method for generating a mass spectra for the compounds present in the sample. Additionally, numerous embodiments will obtain mass spectra for the sample from already performed analyses that are saved or preserved in a database.

At Step 1002, some embodiments will obtain mass spectra for the desired resource. These desired resource mass spectra are obtained from a database or other source of mass spectra for the desired resource, in numerous embodiments. For example, these mass spectra can represent mass spectra for crude oil, natural gas, or other desired resources.

In various embodiments, a user will define one or more reaction networks that can be utilized to synthesize the compounds present in the sample at Step 1004 of certain embodiments. As noted herein, the reaction network includes defining domains, chemical species, and reactions utilized to synthesize the compound or compounds of interest. For compounds of interest that have multiple methods of synthesis, some embodiments will allow the user to enter all known or possible reaction networks, while other embodiments may only allow a single reaction network at a time for further analysis. Additionally, further embodiments allow the user to provide multiple different conditions or physical properties within the domain for the reaction network, such as temperature, pressure, pH, and any other condition that may affect reactions for the synthesis of the desired resources. Multiple conditions can be set as individual units, where the various parameters (e.g., temperature, pH, and pressure) are set to specific values in some embodiments. For example, in some embodiments, a user can set the temperature to 0° C. and 100° C. or pH to 4, 7, and 10. In additional embodiments, a user can set the conditions as ranges, such that temperature can be set to 0° C. to 100° C. or pH of 4-10.

At Step 1006, systems and methods will assess the reaction networks by querying databases for reaction and chemical constants to generate predicted mass spectra and/or predicted proportions of chemical species and isotopologues as described herein, in various embodiments. Additionally, if the reaction conditions are set to include multiple conditions, whether discrete values or ranges, more embodiments will provide multiple versions of the mass spectra covering the multiple conditions set for the domain.

Once the predicted mass spectra and proportions are generated, various embodiments will compare the predictions against the mass spectra of the desired resource to identify conditions that would generate the desired resource at Step 1008. Additionally, by comparing the predicted mass spectra to the sample in some embodiments, the conditions that existed to synthesize the compounds in the sample would also be known.

At step 1010, some embodiments quantify the differences in conditions that would generate the desired resource with the conditions that gave rise to the compounds in the sample. Based on differences in the conditions between the sample and the desired resource, various embodiments will provide quantifiable differences that will generate the desired resource over the sample. For example, if the difference between the sample and the desired resource is an increase in pressure, this difference is quantified by the conditions that give rise to those specific mass spectra. As such, a deeper location in the earth may be more appropriate for the formation of the desired natural resource. As such, identifying these differences will provide a person seeking the desired natural resource a better location to find the desired natural resource.

At Step 1012 of various embodiments, the desired natural resource is extracted through appropriate means, such as mining, drilling, or fracking, based on the difference(s) discovered in Step 1010.

### Exemplary Embodiments

Experiments were conducted to demonstrate the capabilities of the assays and inhibitors in accordance with embodiments. These results and discussion are not meant to be limiting, but merely to provide examples of operative devices and their features.

### Example 1: Predicting the Formation of Calcium Carbonate

Background:

An embodiment of a system in accordance with this disclosure was used to predict mass spectra for the formation of calcium carbonate.

Methods:

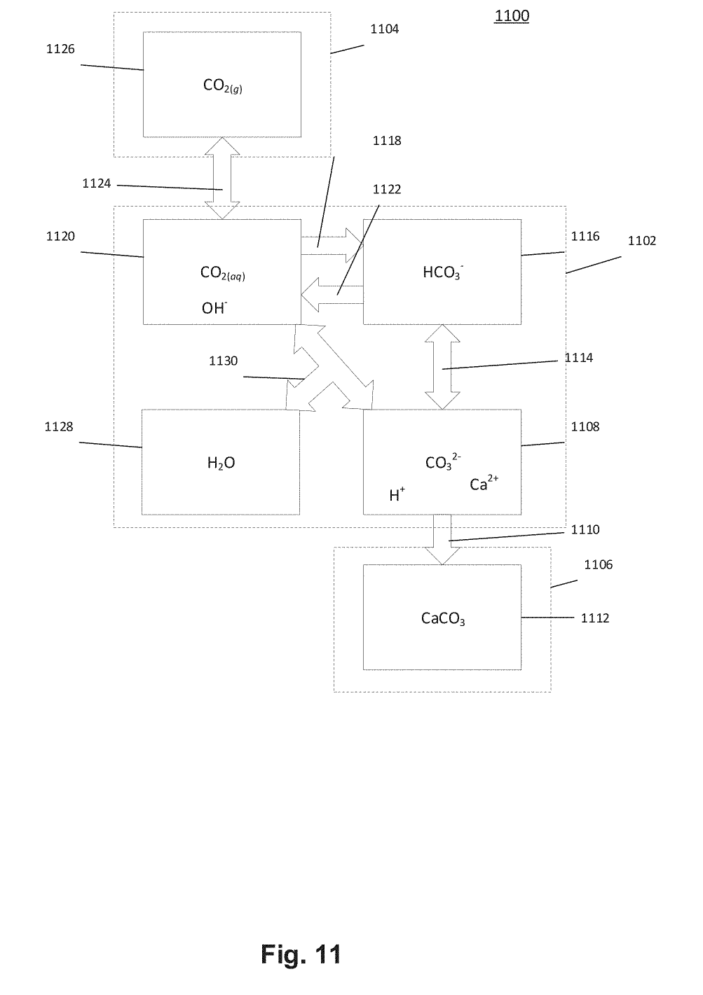

FIG. 11 how reaction networks are defined in a graphical user interface (GUI) 1100 of some embodiments. This example contains three defined domains 1102, 1104, and 1106, which have different conditions, such as temperature, pH, etc., as described herein. This example further includes six boxes (e.g., 1108) representing reactants and products for six individual reactions (e.g., 1110).

### Defining a Reaction Network and Chemical Species

In this embodiment, CO₃²⁻ is added to the GUI, which populates a first box 1108 to indicate reactants or products as part of one or more reaction. After the first box 1108 populates, Ca²⁺ is added to the box. A first reaction 1110 is created, which populates a second box 1112. The first reaction 1110 is set to be an irreversible reaction to create calcium carbonate (CaCO₃), which is added to the second box 1112 in the third domain 1106, as calcium carbonate precipitates out of the aqueous solution.

A second reaction 1114 is added to create CO₃²⁻ from HCO₃_{^{−}} in an equilibrium reaction, which populates a third box 1116, which includes HCO₃_{^{−}} and places H⁺ in the first box 1108 as a product.

A third reaction 1118, which is irreversible, is added to the interface to create HCO₃_{^{−}} from aqueous carbon dioxide (CO_{2(aq)}) and hydroxide (OH⁻), which populates a fourth box 1120. A fourth reaction 1122, which is irreversible, is placed to show the reverse reaction to create aqueous carbon dioxide and hydroxide from HCO₃_{^{−}} in the third box 1116. As the reactants and products are already present, no additional boxes are populated.

A fifth reaction 1124 is placed to show the equilibrium reaction between aqueous carbon dioxide and gaseous carbon dioxide (CO_{2(g)}). By placing this reaction, a fifth box 1126 is populated and includes gaseous carbon dioxide. This fifth box is placed in the second domain 1104, since the conditions differ (gaseous versus aqueous).

At this situation, the reactions are not balanced for a lack of a reactants and products of H⁺ and OH⁻ in the GUI 1100. To solve the stoichiometry, a sixth box 1128 is added including water (H₂O). However, the reaction for water requires interactions between the first, fourth, and sixth boxes (1108, 1120, and 1128). A sixth reaction 1130 showing this interaction is placed in the first domain 1102.

At this stage, the current inventory of chemical species placed in the GUI 1100 is listed in Table 1:

TABLE 1
Inventory of Chemical Species

CO_{2(g)}
CO_{2(aq)}
H₂O
OH⁻
H⁺
HCO₃⁻
CO₃²⁻
Ca²⁺
CaCO₃

Further, the reactions present in GUI 1100 are listed in Table 2:

TABLE 2
Inventory of Reactions

| Reaction Number | Type of Reaction | Equation |
|---|---|---|
| R1 | Irreversible | CO₃²⁻ + Ca²⁺ → CaCO₃ |
| R2 | Equilibrium | CO₃²⁻ + H⁺ ↔ HCO₃⁻ |
| R3 | Irreversible | CO_{2(g)} + OH⁻ → HCO₃⁻ |
| R4 | Irreversible | HCO₃⁻ → CO_{2(aq)} + OH⁻ |
| R5 | Equilibrium | CO_{2(g)} ↔ CO_{2(aq)} |
| R6 | Equilibrium | H₂O ↔ OH⁻ + H⁺ |

As noted above, the three different domains (1102, 1104, and 1106) differ due to the domains occurring in aqueous (e.g., first domain 1102), gaseous (e.g., second domain 1104), and solid (e.g., third domain 1106) phases. In the aqueous first domain 1102, the user can define certain functions, such as activity of water and pH. In this example, the water was set to an activity of 1, while pH was selected was set at 7. Additionally, the amounts available for each reaction can be set, so the gaseous phase can set the carbon dioxide amounts available to an infinite amount. These settings are user imposed constraints on the reaction network. Further, whether to run the model as steady state or time-dependent is selected, depending on the option for the user. If time-dependent is selected, a time is also selected. In this embodiment, steady state was selected and allowed to run until a final result is produced.

The isotopic compositions are then set. The initial isotopic compositions are set. In the present example, the initial composition of isotopes were set from the initial reactants, as listed in Table 3:

TABLE 3
Initial Isotopic Composition

CO_{2(g)}
CO_{2(aq)}
H₂O
OH⁻
H⁺
Ca²⁺

After setting the parameters identified in the GUI 1100 for the reaction network, the system queries chemical and reaction databases (e.g., FIG. 3, 310) to identify equilibrium constants for all reversible/equilibrium reactions and rate law expressions for irreversible reactions. This model produces predicted mass spectra for the compounds identified as products in the reaction network.

Conclusion:

It is possible to create computing systems that are easy to use to generate mass spectra.

### DOCTRINE OF EQUIVALENTS

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference

What is claimed is:

1. A system to generate isotopic structure and mass spectra predictions comprising:
a processor; and
a memory, wherein the memory contains instructions that when executed by the processor direct the processor to:
obtain a reaction network and a plurality of chemical species, wherein the reaction network includes at least one chemical reaction, and each chemical specie in the plurality of chemical species is a chemical compound;
impose constraints on the plurality of chemical species and the reaction network, wherein the constraints are obtained by querying a database of reaction constants and chemical constants;
calculate a mass spectra prediction based on the reaction network, chemical species, and constraints; and
produce an isotopic structure prediction and a visualized mass spectrum prediction based on the calculated mass spectra prediction.

2. The system of claim 1, wherein the chemical constants include constants for a plurality of chemical species, wherein the constants for a plurality of chemical species include at least one of the group consisting of: number of atoms in the plurality of chemical species, type of atoms in the plurality of chemical species, 13 factors for the plurality of chemical species, number of bonds in the plurality of chemical species, type of bonds in the plurality of chemical species, and kinetic isotope effect for the plurality of chemical species.

3. The system of claim 1, wherein the reaction constants include constants for plurality of chemical reactions, wherein the constants include at least one of the group consisting of: K_{eq}, type of reaction, rate law constants.

4. The system of claim 1, wherein the reaction network contains a domain, wherein the domain represents a physical space having at least one physical property, wherein the at least one physical property is selected from the group consisting of: specified volume, surface area, temperature, pressure, pH, Eh, and oxygen fugacity.

5. The system of claim 1, wherein the instructions further direct the processor to impose initial constraints on the reaction network based on activity of the plurality of chemical species.

6. The system of claim 1, wherein the instructions further direct the processor to impose initial constraints on the reaction network based on initial abundance of the plurality of chemical species.

7. The system of claim 6, wherein the instructions further direct the processor to:
query a user whether to run a time-varying solution or steady state solution; and
impose a further constraint to change the abundance of the plurality of chemical species over time.

8. The system of claim 7, wherein the instructions further direct the processor to query a database of equilibrium partition functions to identify specific equilibrium partition functions for the plurality of chemical species, wherein the partition functions define equilibrium proportions of singly and doubly substituted isotopologues of various chemical species.

9. The system of claim 8, wherein the instructions further direct the processor to calculate an equilibrium partition function for at least one of the plurality of chemical species.

10. The system of claim 8, wherein the instructions further direct the processor to:
obtain initial isotopic contents for the plurality of chemical species;
combine the initial isotopic contents with the abundance of the plurality of chemical species;
calculate isotope exchange equilibria for the plurality of chemical species based on the specific partition functions and the combined initial isotopic contents and the abundance of the plurality of chemical species;
determine kinetic isotope effects of the plurality of chemical species; and
determine proportions of isotopologues of the plurality of chemical species.

11. The system of claim 10, wherein the instructions further direct the processor to:
query a database of mass spectra to identify a mass spectra for each of the plurality of chemical species;
calculate a mass spectra prediction based on the identified mass spectra and the proportions of isotopologues; and
generate the visualized mass spectra prediction based on the calculated mass spectra prediction.

12. The system of claim 11, wherein the instructions further direct the processor to:
define a mass resolution of the mass spectra prediction; and
recalculate the mass spectra prediction based on the defined mass resolution.

13. The system of claim 11, wherein the instructions further direct the processor to:
query a database of reference standards to identify a reference standard compatible with the identified mass spectra; and
produce the compatible reference standard.

14. The system of claim 13, wherein the visualized mass spectra prediction is generated by ratioing the calculated mass spectra prediction to a mass spectrum of the compatible reference standard.

15. The system of claim 1, further comprising a graphical user interface for accepting input from a user and producing the visualized mass spectra prediction.

16. The system of claim 1, further comprising four graphical user interfaces, wherein a first graphical user interface is used to input the reaction network, a second graphical user interface is used to input the plurality of chemical species, a third graphical user interface is used to output the isotopic structure prediction, and a fourth graphical user interface is used to output the visualized mass spectrum prediction.

17. The system of claim 1, wherein the obtained reaction network and the obtained plurality of chemical species are received over a network from a user device.

18. The system of claim 1, wherein the isotopic structure prediction and the visualized mass spectrum prediction are provided to a user device over a network.

19. A method of extracting oil comprising:
obtaining a mass spectrum from a sample obtained from a geologic source to identify at least one compound present in the sample;
defining a reaction network to synthesize the at least one compound present in the sample and a desired compound, wherein the reaction network contains a domain possessing a physical property which has multiple settings;
generating a plurality of visualized mass spectra predictions based on the reaction network, wherein the plurality of visualized mass spectra predictions represent the reaction network at each of the multiple settings of the physical property;
identifying a first setting and a second setting from the reaction network, wherein the first setting identifies a physical property condition that led to synthesis of the desired compound and the second setting identifies a physical property condition that led to synthesis of the at least one compound present in the sample;
quantifying a difference in the at least one physical property that led to synthesis of the desired compound over the at least one compound present in the sample; and
extracting the desired compound based on the quantified difference.

20. The method of claim 19, wherein the extracting step is accomplished by one of the group consisting of mining, drilling, and fracking.
